(19) United States
(12) Patent Application Publication
(10) Pub. No.: US 2005/0120734 A1
(43) Pub. Date: Jun. 9, 2005

(54) Induction of hypothermia by infusion of saline slush

(75) Inventor: Steve A. Yon, San Diego, CA (US)

Correspondence Address: Karin L. Williams;Mayer Fortkort & Williams, 251 North Ave. West, 2nd Floor, Westfield, NJ, 07090, US

(73) Assignee: Innercool Therapies, Inc.

(21) Appl. No.: 11/006,229

(22) Filed: Dec. 6, 2004

Related U.S. Application Data
(60) Provisional application No. 60/527,109, filed on Dec. 4, 2003.

(51) Int. Cl. F25D 13/06; C09K 3/18; A61F 7/00; F25D 13/00

(52) U.S. Cl. 62/330; 252/70; 607/105

(57) ABSTRACT

The invention provides techniques for producing a saline slush for induction of therapeutic hypothermia. Embodiments of the invention provide a gain of hypothermic therapies over the use of chilled saline alone.

### BACKGROUND OF THE INVENTION

Therapeutic hypothermia has been induced in the past by direct venous or arterial infusion of chilled solutions, typically 0.9% saline which is available in most clinical settings. If this fluid is injected at a temperature near 0 C., the effective ‘cooling power’ applied to perfused tissue is directly proportional to body temperature and the rate of infusion. If the mean tissue temperature is 37 C. and the chilled fluid is infused at 1 L/hr., the body will supply roughly 42 Watts to raise the temperature of the infused fluid. Increasing the mass flux of infusate will result in a greater rate of heat extraction from perfused tissue, but there is a limit to the rate and ultimate amount of fluids that may be safely infused. This requirement is made stricter by certain clinical conditions such as AMI. Heart attack patients are not typically given large amounts of fluid in order to minimize stress to which the heart is subjected.

### SUMMARY OF THE INVENTION

Recent research suggests, however, that reducing myocardial temperature prior to reestablishing blood flow to the ischemic tissue can substantially reduce the permanent infarct size. If the total volume of fluid which can be administered to AMI patients is limited, then raising the effective heat capacity of the infused fluid may allow effective application of therapeutic hypothermia. If the infused fluid were a slush, or a mixture of water ice and a saline solution chosen so that the bulk composition matches that of 0.9% saline, then in addition to the heat capacity of the liquid saline, the total heat absorbed during equilibration with body temperature would include the latent heat available in the infused ice. This technique has the potential to significantly increase the effective ‘cooling power’ available by infusion of chilled fluids. The invention provides techniques for producing a saline slush for induction of therapeutic hypothermia.

### BRIEF DESCRIPTION OF THE DRAWINGS

FIG. 1 shows the binary phase diagram of the simple NaCl—H₂O system at ambient pressure.

FIG. 2 shows the ice content (wt %) of a 1% saline solution as a function of temperature.

FIG. 3 shows the composition of the residual fluid as a function of ice content.

FIG. 4 shows the effective cooling power as a function of Wₛ for two infusion rates, assuming a body temperature of 36 C.

### DETAILED DESCRIPTION

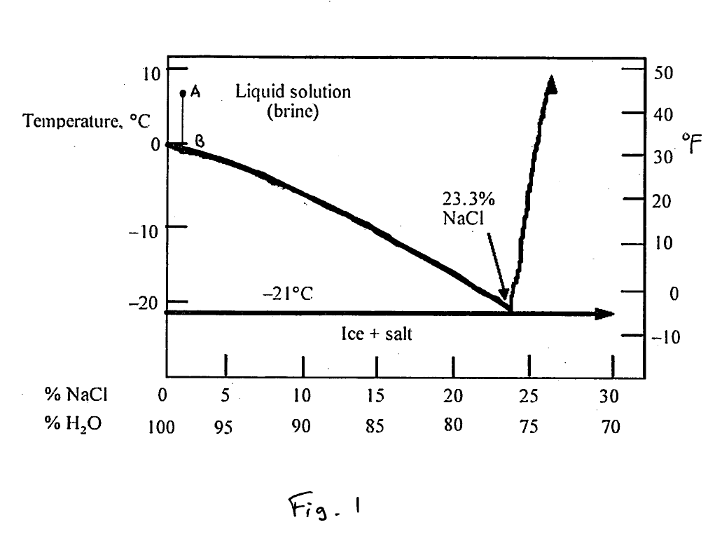

Saline solutions, modeled in the simplest form by dissolving a specified weight percent of sodium chloride (salt) in a known mass of pure water, are eutectic systems. The binary phase diagram of the simple NaCl—H₂O system at ambient pressure is shown in FIG. 1. The eutectic point, which is the lowest temperature at which any liquid may exist in equilibrium, occurs at −21 C. in association with a fluid containing roughly 23% (by wt.) NaCl. Any saline solution will yield a residual fluid with this composition as the temperature is reduced to −21 C. Beginning with a liquid saline solution of known composition, e.g. 1% to approximate the clinical 0.9% solution, and a temperature above the liquidus (the curved line connecting the eutectic point with the freezing point of pure water at 0 C.) represented by point ‘A’ in FIG. 1, reducing temperature yields a single phase until the temperature intersects the liquidus at point ‘B’. At this temperature, two phases exist in equilibrium. Pure water ice forms and the saline concentration in the residual fluid is incrementally increased. As temperature is reduced below the liquidus, pure water ice continues to precipitate and the composition of the residual fluid follows the liquidus. The fact that production of each incremental mass of water ice yields a residual fluid which is slightly increased in saline composition (since the precipitated solid contains none of the NaCl) results in a 2-phase system which is stable (i.e. the weight % of solid vs. liquid) at any temperature between the liquidus and −21 C. The amount of solid vs. liquid is calculated by the lever rule. If c is the composition of the residual fluid, then the weight % of ice at a fixed temperature is given by

$${W}_{s} = \frac{c - 1}{c}$$

assuming bulk composition of 1% saline as previously postulated.

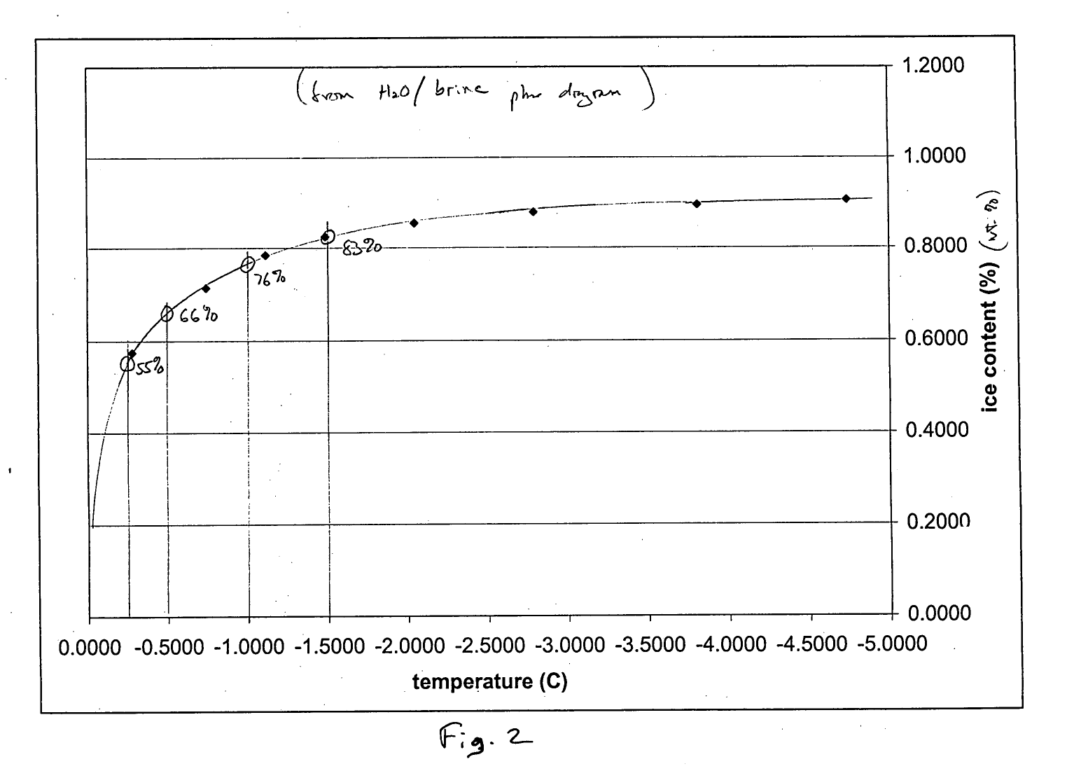

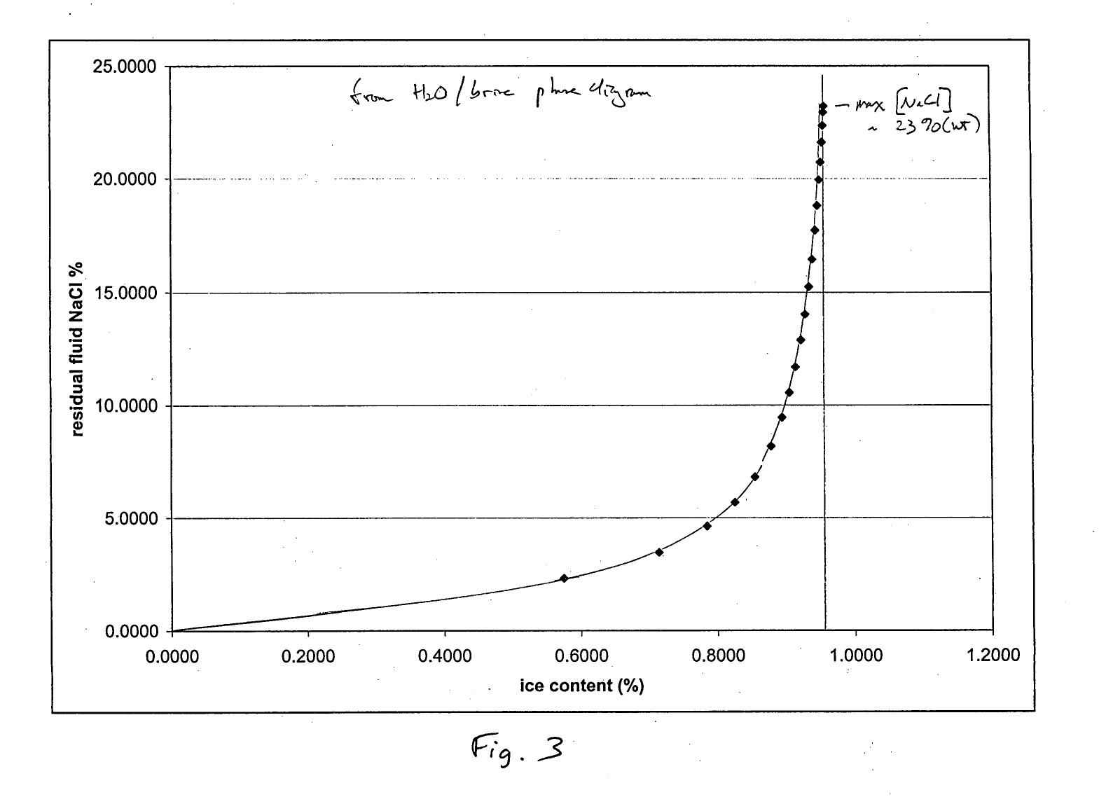

Using the phase diagram shown in FIG. 1, the ice content (wt %) of a 1% saline solution as a function of temperature is shown in FIG. 2. The ice content of the solution in FIG. 2 at temperatures above −0.25 C. is not shown to avoid ambiguity in the phase diagram used in its construction. As temperature is reduced, the solid (ice) content of the system increases to a maximum of approximately 95% as temperature approaches the −21 C. minimum. The composition of the residual fluid at any temperature is defined by the liquidus in the phase diagram (FIG. 1) or alternatively as a function of ice content as shown in FIG. 3. For example, with an ice content of 50% (by weight), the residual fluid contains approximately 2% (by weight) salt.

Referring to FIG. 2, this solution would be stable at approximately −0.25 C. The salt concentration of the residual fluid in this example is significantly greater than that of isotonic (0.9%) saline, and it should not be infused without the associated water ice which renders the bulk infusate isotonic. In summary, the eutectic nature of saline solutions simplifies the production of liquid-ice slush since the composition of the system at any temperature (i.e. in terms of weight % ice) is a deterministic function of the solution temperature. Production of an isotonic infusate with a specific weight % ice is accomplished by allowing a volume of isotonic saline solution to equilibrate at the required temperature as obtained from FIG. 2.

Energy Absorption Available in Saline Slush:

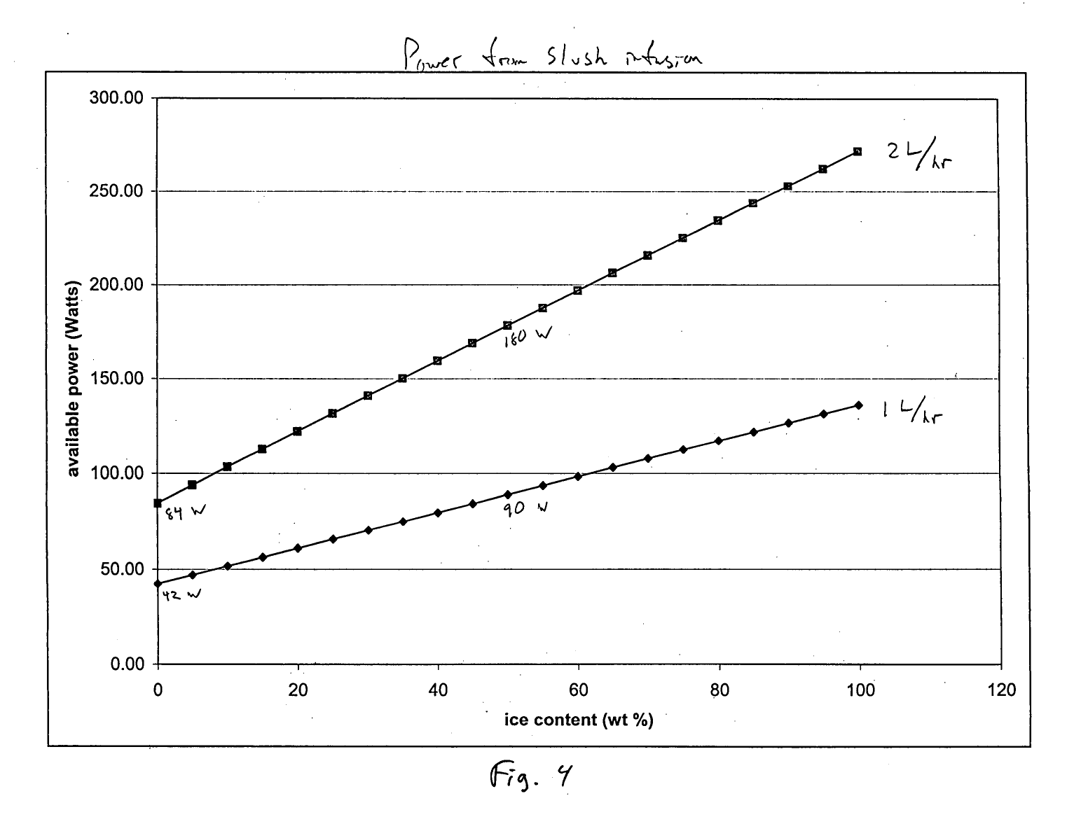

Including the latent heat of fusion available in the mass fraction of ice present in a saline slush, the power required to raise the temperature of a stream of slush, as would occur during infusion into a body, is given by
P=\dot{m}(CₚΔT+Wₛh_{f})
in which \dot{m} is the mass flux of infusate, Cₚ is the specific heat capacity of the liquid infusate, Wₛ is the mass fraction of ice, h_{f} is the latent heat of fusion of ice, and ΔT is the temperature difference between the infusate and the body. This effective cooling power is shown in FIG. 4 as a function of Wₛ for two infusion rates, assuming a body temperature of 36 C. In both cases, the maximum theoretical gain over the power available using chilled saline (Wₛ=0) is slightly more than a factor of 3. Clinically, infusion of a slush with Wₛ>0.5 may not be practical, which further limits the gain relative to chilled saline to approximately a factor of 2. Using clinically acceptable infusion rates, cooling power available by slush infusion may not exceed 180 Watts. In addition, the total energy delivered will be limited by the clinically-dictated total infusate volume.

Implementations of the invention may include the following aspects. A temperature-controlled chamber may be employed to ensure the desired ice wt %. A sterilized saline bag with a mixing bar inside the bag (for mechanical agitation to break ice xls) may be employed. A mixing plate inside the temperature controlled chamber with an inflatable cuff to mix/squeeze the slush bag may be employed. A peristaltic pump to move slush may be used so that the bag squeeze does not simply push out fluid. Slush may be removed from the upper surface so that crystals are naturally entrained (since they will float). To this issue, the mixing plate may need to be inclined so that air infusion is unlikely. An air detector may be used to prevent air infusion.

The invention has been described with respect to a number of embodiments. However, the invention is to be limited only by the claims appended hereto.

1. A method for producing phase change particulate saline slurries comprising the steps of:
providing a saline solution with a specified saline content;
cooling said saline solution to a set temperature,
Such that the specified saline content and the set temperature are chosen to correspond to a desired point on the binary phase diagram of the NaCl—H₂O system at ambient pressure, such that an ice slurry of a desired nature is created.

2. A method for producing phase change particulate saline slurries as recited in claim 1, therein said steps of providing a saline solution provides a saline solution in a range between about 0.5% to 6.0%.

3. A method for producing phase change particulate saline slurries as recited in claim 1, further comprising the step of cycling the temperature about the set temperature to smooth particles in the ice slurry.

4. Apparatus for producing phase change particulate saline slurries comprising:
a container for containing a saline solution;
a cooler for cooling said saline solution to a set temperature;
means for controlling the temperature of said saline solution.

5. A method of treating a patient comprising:
administering to the patient a phase-change particulate slurry internally, the slurry containing a saline solution with a specified saline content that has been cooled to a set temperature such that the specified saline content and the set temperature are chosen to correspond to a desired point on the binary phase diagram of the NaCl—H₂O system at ambient pressure; and
continuing to administer the slurry until a state of hypothermia is induced in the patient.

6. The method of claim 5, wherein the phase-change particulate ice slurry further comprises a 0.9% sodium chloride solution.

7. The method of claim 5, wherein the sodium chloride solution further comprises a concentration of sodium chloride between about 0.5% to 3.0%.

8. The method of claim 5, wherein the sodium chloride solution further comprises a concentration of sodium chloride of about 1.0%.

9. The method of claim 5, further comprising administering the phase-change particulate slurry to the patient intravascularly.

10. The method of claim 9, further comprising administering the phase-charge particulate slurry into the aorta of the patient.

11. The method of claim 5, further comprising administering the phase-change particulate slurry into the gastrointestinal tract of the patient.

12. A phase-change particulate ice slurry for inducing hypothermia in a patient comprising a medical grade saline solution and ice particles, the saline concentration being in a range between about 0.5% to 3.0%.

13. The phase-change particulate slurry of claim 12, wherein the saline solution is sodium chloride.

14. The phase-change particulate slurry of claim 13, wherein the percentage of ice particles in the slurry is between about 5% and 40%.

15. An apparatus for inducing hypothermia in a patient comprising:
(a) a liquid vessel containing a phase-change particulate slurry, the slurry containing a saline solution with a specified saline content that has been cooled to a set temperature such that the specified saline content and the set temperature are chosen to correspond to a desired point on the binary phase diagram of the NaCl—H₂O system at ambient pressure;
(b) means connected to the liquid vessel for delivering the phase-change particulate slurry internally to the patient;
(c) means for continuously administering the phase-change particulate slurry from the liquid vessel to the patient until a state of hypothermia is induced in the patient.

16. The apparatus of claim 15, wherein the phase-change particulate slurry further comprises a saline solution and ice particles, the saline concentration being in a range between about 0.5% to 3.0%.

17. The apparatus of claim 16, wherein the saline solution is sodium chloride.

18. The apparatus of claim 17, wherein the saline concentration is about 0.9%.

19. The apparatus of claim 16, wherein the percentage of ice particles in the phase-change particulate slurry is between about 5% and 40%.

20. The apparatus of claim 16, wherein the saline concentration is about 0.9%.

21. The apparatus of claim 15, further comprising means for cooling the liquid vessel.

22. The apparatus of claim 15, further comprising means for mixing contents within the liquid vessel.

23. An apparatus for inducing hypothermia in a patient comprising:
(a) a liquid vessel containing a phase-change particulate slurry, the slurry containing a saline solution with a specified saline content that has been cooled to a set temperature such that the specified saline content and the set temperature are chosen to correspond to a desired point on the binary phase diagram of the NaCl—H₂O system at ambient pressure;
(b) means connected to the liquid vessel for delivering the phase-change particulate slurry internally to the patient, whereby the phase-change particulate slurry is delivered from the liquid vessel to the patient until a state of hypothermia is induced in the patient.

24. The apparatus of claim 23, further comprising means for cooling the liquid vessel.

25. The apparatus of claim 23, further comprising means for mixing contents within the liquid vessel.

26. The apparatus of claim 15, wherein the phase-change particulate slurry comprises a saline concentration of about 1.0%.

27. An apparatus for administering a phase-change particulate slurry to a patient comprising:
(a) a liquid vessel containing a phase-change particulate slurry, the slurry containing a saline solution with a specified saline content that has been cooled to a set temperature such that the specified saline content and the set temperature are chosen to correspond to a desired point on the binary phase diagram of the NaCl—H₂O system at ambient pressure;
(b) a delivery device including a flexible, tubular member connected to the liquid vessel for delivering the phase-change particulate slurry internally to the patient, the flexible, tubular member having a first end and a second end, the first end insertable inside a patient, and the second end connected to the liquid vessel and in fluid communication with the liquid vessel, whereby the phase-change particulate slurry is delivered from the liquid vessel through the flexible, tubular member and into the patient until a state of hypothermia is induced in the patient.

28. The apparatus of claim 27, wherein the flexible tubular member is adapted to be inserted into the gastrointestinal tract of the patient.

29. The apparatus of claim 27, wherein the flexible tubular member is adapted to be inserted into the patient intravascularly.

30. The apparatus of claim 27, wherein the phase-change particulate slurry comprises a saline concentration of about 0.9%.
